(19) United States
(12) Patent Application Publication
(10) Pub. No.: US 2005/0080122 A1
(43) Pub. Date: Apr. 14, 2005

(54) Anti-viral compounds

(76) Inventors: David Andrew Anderson, Brunswick (AU); Elena Vladimirovna Gazina, Thornbury (AU)

Correspondence Address: SCULLY SCOTT MURPHY & PRESSER, PC, 400 GARDEN CITY PLAZA, GARDEN CITY, NY, 11530, US

(21) Appl. No.: 10/503,324

(22) PCT Filed: Jan. 30, 2003
(86) PCT No.: PCT/AU03/00093

(30) Foreign Application Priority Data
Jan. 31, 2002 (AU) PS0228

(51) Int. Cl. A61K 31/4172; A61K 31/137

(52) U.S. Cl. 514/396; 514/649

(57) ABSTRACT

The present invention relates generally to compounds useful in the amelioration of symptoms associated with viral infection. More particularly, the present invention relates to the use of compounds which exhibit a physiological effect on membranous and/or transmembranous structures on or in a cell and which directly or indirectly reduce or inhibit or otherwise prevent viral infection, processing and/or release from the cell. Even more particularly, the present invention contemplates the use or one or more compounds which modulate at least one host cell ion channel in the prophylaxis, treatment and/or symptomatic relief of viral infection in vertebrate animals and in particular in human subjects. The compounds may be provided alone or in combination with other compounds such as those which block or inhibit or at least impair ion channeling. A preferred embodiment of the present invention is the use of the aforementioned anti-viral compounds in the therapeutic management of vertebrate animals including humans, to prevent, reduce or treat infection by certain species of the Picornaviridae family of viral pathogens such as but not limited to Rhinovirus or Enterovirus species.

### BACKGROUND OF THE INVENTION

1. Field of the Invention

The present invention relates generally to compounds useful in the amelioration of symptoms associated with viral infection. More particularly, the present invention relates to the use of compounds which exhibit a physiological effect on membranous and/or transmembranous structures on or in a cell and which directly or indirectly reduce or inhibit or otherwise prevent viral infection, processing and/or release from the cell. Even more particularly, the present invention contemplates the use of one or more compounds which modulate at least one host cell ion channel in the prophylaxis, treatment and/or symptomatic relief of viral infection in vertebrate animals and in particular in human subjects. The compounds may be provided alone or in combination with other compounds such as those which block or inhibit or at least impair ion channeling. A preferred embodiment of the present invention is the use of the aforementioned anti-viral compounds in the therapeutic management of vertebrate animals including humans, to prevent, reduce or treat infection by certain species of the Picornaviridae family of viral pathogens such as but not limited to Rhinovirus or Enterovirus species.

2. Description of Thee Prior Art

Bibliographic details of the publications referred to by author in this specification are collected at the end of the description.

Reference to any prior art in this specification is not, and should not be taken as, an acknowledgment or any form of suggestion that this prior art forms part of the corninon general knowledge in any country.

The development of medicines to treat viral pathogens has been a goal of health researchers worldwide for many years, but despite close attention to the task there remains a need for effective anti-viral drugs for the efficacious therapeutic management of a vast number of viral-mediated illnesses.

There are numerous problems which are encountered in the search for drugs that will effectively inhibit the spread of viruses in a host organism. Briefly, these include the ability of many viruses to avoid activating the immune system's detection mechanism, the capacity of viruses to replicate and spread prior to being detected by the immune system and the high mutation rate of viral proteins.

The Picomaviridae family is a ubiquitous group of viral pathogens that causes the most common, and often serious, viral-mediated illnesses in humans. The range of diseases caused by this group includes asceptic meningitis, poliomyelitis, certain types of myocarditis as well as rhinovirus infection. Rhinoviruses cause over 80% of all cases of acute nasopharyngitis (the common cold) [Monto et al., Clin. Ther. 10: 1615-27, 2001], and hence are the cause of the one respiratory infection which creates the most restriction of activity and necessitates the greatest number of physician consultations per year in Western nations.

The Picornaviridae family is also of veterinary significance: the Aphthovirus genus is responsible for the recent, economically devastating foot-and-mouth disease in livestock animals.

Hence, due to its ability to infect humans and livestock animals alike, this group of pathogens represents a significant economic liability worldwide.

New drugs such as Pleconaril have been developed with the specific purpose of treating rhinovirus- and other types of picornavirus infections, but a significant drawback of these drugs is that there is the potential for the formation of Pleconaril-resistant viral strains (Turner, Antiviral Res. 49(1): 1-14, 2001). This is a consequence of the high capacity of viruses to mutate and overcome the effects of anti-viral compounds that have been designed to inhibit one or more steps in the process of the virus's entry into a host cell and its subsequent replication in the cell.

Research has found that infection by certain members of the Picomaviridae family cause alterations in the intracellular ion levels of infected cells. In particular, poliovirus and Coxsackievirus cause increases in the cytoplasmic concentration of calcium (Irurzum et al., J. Virol. 69(8): 5142-6, 1995; van Kuppeveld et al., EMBO J. 16(12): 3519-32, 1997) and encephalomyelitis virus infection as well as poliovirus infection have both been found to disrupt cellular sodium and potassium-ion homeostasis (Egberts et al., J. Virol. 22(3): 591-7, 1977; Nair, J. Virol. 37(1): 268-73,1981; Nair et al., J. Virol. 31(1): 184-9, 1979). The net result of these alterations in ionic transport appears to be an influx of sodium and/or calcium ions into the cell cytoplasm.

In accordance with the present invention, compounds are identified which alter the permeability of ion channels in the host cell and, surprisingly, found that these compounds were effective in controlling the replication and/or spread of viruses and in particular of members of the Picornaviridae family.

### SUMMARY OF THE INVENTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

The present invention provides a method for the treatment or prophylaxis of viral infection in vertebrate animals such as but not limited to humans, livestock animals, avian species, companion animals, and laboratory test animals. The compounds are generally defined by their ability to modulate the activity of a membranous or transmembranous structure which permits passage of ions into or out of a vertebrate animal cell.

In a preferred embodiment, these membranous or transmembranous structures are referred to as ion channels and the preferred compounds are generally regarded as “ion channel blockers” or “ion channel modulators”. Such compounds are proposed inter alia to alter the functional properties of an ion channel such as creating an open-channel state (complete activation), partial activation, inhibition or total blockage of the ion channel.

The compounds contemplated in the present invention are represented in Formulae I through IV as herein defined. The preferred compounds are Verapamil (Formula V), Econazole (Formula VI), Benzamil (Formula VII) and 5-(N-ethyl-N-isopropyl)amiloride [EIPA] (Formula VIII) and Amiloride (Formula IX), the parent compound of EIPA. Reference to all such compounds include pharmaceutical salts thereof as well as derivatives thereof.

The compounds may be administered singularly or in combination with each other or with compounds having antiviral properties, ion channel-blocking properties or compounds which otherwise facilitate amelioration of the symptoms of viral infection.

The compounds may, therefore, be in the form of a pharmaceutical composition comprising the compound and one or more pharmaceutically acceptable carriers and/or diluents.

The viruses are generally members of the Picomaviridae family such as but not limited to species of Rhinovirus or Enterovirus.

In a preferred embodiment, the compounds are administered to a subject for a time and under conditions sufficient to ameliorate the symptoms of viral infection or to prevent or reduce viral infection.

The present invention further provides for the use of a compound of general Formulae I-IV or more specifically Verapamil, Econazole, Benzamil and/or EIPA as well as its parent compound Amiloride in the manufacture of a medicament for the treatment or prophylaxis of viral infection in a vertebrate animal such as a human. EIPA and Verapamil were determined to be particularly effective against Rhinovirus. Amiloride and Benzamil are particularly effective against Enterovirus.

Reference to the “compounds” of the present invention means the general compounds of Formulae I-IV and the specific compounds in Formulae V-IX. The term “compounds” also encompasses “chemical agents” as well as “therapeutic agents” and “active ingredient” and “active”.

### BRIEF DESCRIPTION OF THE FIGURES

FIG. 1 is a graphical representation of the effect of 5-(N-ethyl-N-isopropyl)amiloride [EIPA] on Rhino 2 virus production and HeLa cells. Virus production (-Δ-) and cell metabolism (-o-) were measured against concentration of EIPA (in μM).

FIG. 2 is a graphical representation of the effects of Verapamil on Rhino 14 virus production and HeLa cells metabolism. Virus product (-Δ-) and cell metabolism (-o-) were measured against concentration of EIP (in EM).

FIG. 3 is a graphical representation of the effects of EIPA on Rhino 2 virus production and HeLa cells. Virus production (-Δ-) and cell metabolism (-o-) were measured against Verapamil (in EM).

FIG. 4 is a graphical representation of the effects of Verapamil on Rhino 14 virus production and HeLa cells. Virus production (-Δ-) and cell metabolism (-o-) were measured against Verapamil (in EM).

FIG. 5 is a graphical representation of the effects of Amiloride on Coxsackievirus B3 production and HeLa cells. Virus production (-Δ-) and cell metabolism (-o-) were measured against Amiloride (in μM).

FIG. 6 is a graphical representation of the effects of Benzamil on Coxsackievirus B3 production and HeLa cells. Virus production (-Δ-) and cell metabolism (-o-) were measured against Benzamil (in μM).

### DETAILED DESCRIPTION OF THE INVENTION

The present invention is predicated in part on the use of compounds which exhibit a physiological effect on membranous or transmembranous structures on or in a cell to reduce the virulence of a viral pathogen for the prophylaxis, treatment and/or symptomatic relief of viral infections in vertebrate animals and in particular humans. A vertebrate animal includes livestock animals, avian species, companion animals, laboratory test animals as well as humans. Humans are particularly preferred.

The term “virulence” in this context includes the ability of the virus to undergo processing in a host cell to generate virus particles.

The term “processing” includes attachment and penetration of viral particles or viral nucleic acid molecules into or onto a cell, viral nucleic acid replication, synthesis of viral-derived proteins and assembly and release of viral particles.

The term “pathogen” includes any virus, whether generally considered pathogenic or not, which causes or at least facilitates a level of infection which induces symptoms of infection.

A “symptom” includes a visible symptoms such as ill health or physical signs of infection or infection identified by, for example, immunological testing.

Accordingly, one aspect of the present invention contemplates a method for ameliorating the effects of Picomaviridae infection in a vertebrate animal said method comprising administering to said animal an effective amount of one or more compounds selected from the compounds of Formulae I-IV or a parent of these compounds: 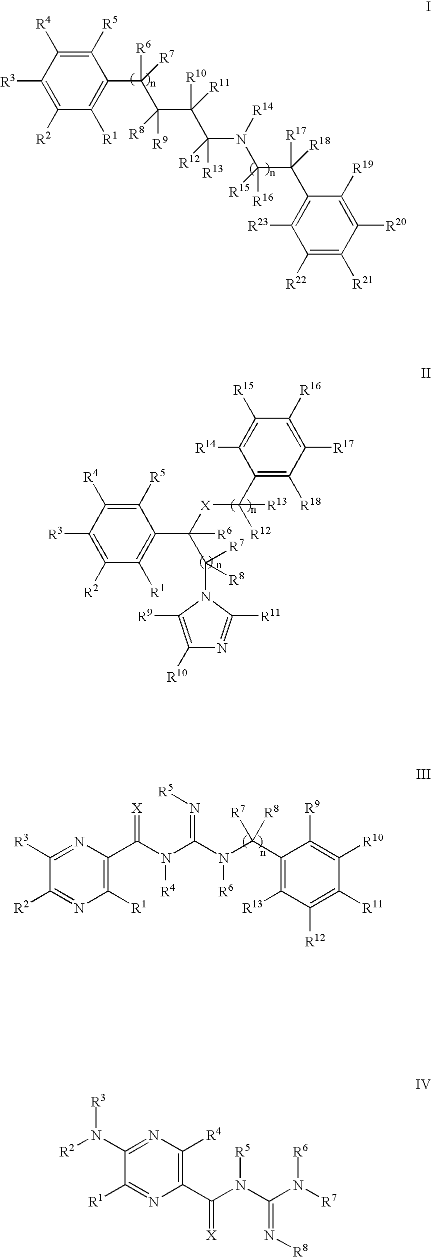
where n is 0-10 atoms and both n and X may be the same or different and each is selected from carbon, oxygen, nitrogen, sulfur, phosphorus, silicon, boron, arsenic and selenium;
R₁ to R₂₃ may be the same or different and each is selected from hydrogen, F, Cl, Br, I, CN, NC, NO₂, CF₃, COR₁, CO₂R₁, OR₁, SR₁, NR₁R₂, N(═O)₂, NR₁OR₂, ONR₁R₂, SOR₁, SO₂R₁, SO₃R₁, SONR₁R₂, SO₂NR₁R₂, SO₃NR₁R₂, P(R₁)₃, P(═O)(R₁)₃, Si(R₁)₃, B(R₁)₂, (C═X)R₁ or X(C═X)R₁ where X is selected from sulfur, oxygen and nitrogen; C₁-C₂₀ alkyl (branched and/or straight chained), C₁-C₂₀ arylalkyl, C₃-C₈ cycloalkyl, C₁-C₁₀ aldoxy, C₁-C₁₀ alkyl carbonyl, C₆-C₁₄ aryl, C₁-C₁₄ heteroaryl, C₁-C₁₄ heterocycle, C₂-C₁₀ alkenyl, C₁-C₁₀ heteroarylalkyl, C₁-C₁₀ alkoxyalkyl, C₁-C₁₀ haloalkyl, dihaloalkyl, trihaloalkyl, haloalkoxy, C₁-C₁₀ [CN, NC, OR₁, SR₁, NR₁R₂, N(═O)₂, NR₁OR₂, ONR₁R₂, SOR₁, SO₂R₁, SO₃R₁, SONR₁R₂, SO₂NR₁R₂, SO₃NR₁R₂, P(R₁)₃, P(═O)(R₁)₃, Si(R₁)₃, B(R₁)₂]alkyl; aryl is C₆-C₁₄ with any mode of substitution containing F, Cl, Br, I, NO₂, CF₃, CN, NC, COR₁, CO₂R₁, OR₁, SR₁, NR₁R₂, N(═O)₂, NR₁OR₂, ONR₁R₂, SOR₁, SO₂R₁, SO₃R₁, SONR₁R₂, SO₂NR₁R₂, SO₃NR₁R₂, P(R₁)₃, P(═O)(R₁)₃, Si(R₁)₃, B(R₁)₂]alkyl. Heteroaryl is oxazolyl, thiazaoyl, thienyl, furyl, 1-isobenzofuranyl, 3H-pyrrolyl, 2H-pyrrolyl, N-pyrrolyl, imidazolyl, pyrazolyl, isothiazolyl, isooxazolyl, pyridyl, pyrazinyl, pyrimidinyl, pyradazinyl, indolizinyl, isoindolyl, indoyl, indolyl, purinyl, phthalazinyl, 1,2,3-triazolyl, 1,2,4-triazolyl, 1,2,3-oxadiazoyl, 1,2,4-oxadiazolyl, 1,2,5-oxadiazolyl, 1,3,4-oxadiazolyl, 1,2,3,4-oxatriazolyl, 1,2,3,5-oxatriazolyl, 1,3,5-triazinyl, 1,2,4-triazinyl, 1,2,3-triazinyl, azepinyl, oxepinyl, thiepinyl, benzofuranyl, isobenzofuranyl, thionaphthenyl, isothionaphthenyl, indoleninyl, 2-isobenzazolyl, 1,5-pyrindinyl, pyrano[3,4-b]pyrrolyl, isoindazolyl, indoxazinyl, benzoxazolyl, anthranilyl, quinolinyl, isoquinolinyl, cinnolinyl, quinazolinyl, naphthyridinyl, pyrido[3,4-b]pyridinyl, pyrido[3,2-b]pyridinyl, pyrido[4,3-b]pyridinyl.

Generally the administration of the compounds is for a time and under conditions sufficient to reduce the amount of virus replication and/or viral release from cells exposed to said compounds. Alternatively, or in addition, the administration is for a time and under conditions for a reduction or amelioration of symptoms of infection.

The compounds of the present invention may also be administered with pharmaceutically acceptable compatible counterions such as salts formed with acids, including but not limited to hydrochloric, sulphuric, acetic, lactic, tartaric, malic and succinic acids.

As used herein the term “alkyl” refers to linear or branched chained saturated, aliphatic hydrocarbon groups having a specific number of carbon atoms. The term “haloalkyl” refers to an alkyl group substituted by at least one halogen. Similarly, the term “haloalkoxy” refers to an alkoxy group substituted by at least one halogen. As used herein the term “halogen” refers to fluorine, chlorine, bromine and iodine. A C₂-C₁₀ alkynyl or C₂-C₁₀ alkenyl may comprise a branched or straight chained aryl and/or heteroaryl attached groups.

As used herein the term “aryl” refers to aromatic carbocyclic ring systems such as phenyl or naphtyl, anthracenyl, especially phenyl. Suitably, aryl is C₆-C₁₄ with mono, di- and tri-substitution containing F, Cl, Br, I, NO₂, CF₃, CN, OR₁, COR₁, CO₂R₁, NHR₁, NR₁R₂, NR₁OR₂, ONR₁R₂, SOR₁, SO₂R₁, SO₃R₁, SONR₁R₂, SO₂NR₁R₂, SO₃NR₁R₂, P(R)₃, P(═O)(R)₃, Si(R₁)₃, BR₂, wherein R₁ and R₂ are as defined for R₁-R₂₃.

As used herein, “haloalkyl” is intended to include both branched and straight-chain saturated aliphatic hydrocarbon groups having the specified number of carbon atoms, substituted with 1 or more halogen (for example—CxFy where x=1 to 3 and y=1 to (2x+1)); “aldoxy” represents an alkyl group with an indicated number of carbon atoms attached through an oxygen bridge; “cycloalkyl” is intended to include saturated ring groups, including mono-, bi- or poly-cyclic ring systems, such as cyclopropyl, cyclobutyl, cyclopentyl, cyclohexyl, cycloheptyl, adamantyl and cyclooctyl. A cycloalkyl includes a “biycloalkyl” includes saturated bicyclic ring groups such as [3.3.0] bicyclooctane, [4.3.0]bicyclononane, [4.4.0] bicyclodecane (decalin), [2.2.2] bibyclooctane, and so forth. “Alkenyl” is intended to include hydrocarbon chains of either a straight or branched configuration and one or more unsaturated carbon-carbon bonds which may occur in any stable point along the chain, such as ethenyl, propenyl, and the like; and “alkynyl” is intended to include hydrocarbon chains of either a straight or branched configuration and one or more triple carbon-carbon bonds which may occur in any stable point along the chain, such as ethynyl, propynyl, and the like. “Alkylcarbonyl” is intended to include an alkyl group of an indicated number of carbon atoms attached through a carbonyl group to the residue of the compound at the designated location. “Alkylcarbonyloxy” is intended to include an alkyl group of an indicated number of carbon atoms attached to a carbonyl group, where the carbonyl group is attached through an oxygen atom to the residue of the compound at the designated location.

“Halo” or “halogen” as used herein refers to fluoro, chloro, bromo and iodo; and “counterion” is used to represent a small, negatively charged species such as chloride, bromide, hydroxide, acetate, sulfate and the like.

The term “substituted” as used herein means that one or more hydrogens on the designated atom is replaced with a selection from the indicated group, provided that the designated atom's normal valency is not exceeded, and that the substitution results in a stable compound.

As used herein the terms “heterocycle”, “heterocyclic”, “heterocyclic systems” and the like refer to a saturated, unsaturated or aromatic carbocyclic group having a single ring, multiple fused rings (for example, bicyclic, tricyclic or other similar bridged ring systems or substituents), or multiple condensed rings, and having at least one heteroatom such as nitrogen, oxygen or sulfur within at least one of the rings. This term also includes “heteroaryl” which refers to a heterocycle in which at least one ring is aromatic. Any heterocyclic or heteroaryl group can be unsubstituted or optionally substituted with one or more groups, as defined above. Further, bi- or tricyclic heteroaryl moieties may comprise at least one ring, which is either completely or partially saturated. Suitable heteroaryl moieties include but are not limited to oxazolyl, thiazaoyl, thienyl, furyl, 1-isobenzofuranyl, 3H-pyrrolyl, 2H-pyrrolyl, N-pyrrolyl, imidazolyl, pyrazolyl, isothiazolyl, isooxazolyl, pyridyl, pyrazinyl, pyrimidinyl, pyradazinyl, indolizinyl, isoindolyl, indoyl, indolyl, purinyl, phthalazinyl, 1,2,3-triazolyl, 1,2,4-triazolyl, 1,2,3-oxadiazoyl, 1,2,4-oxadiazolyl, 1,2,5-oxadiazolyl, 1,3,4-oxadiazolyl, 1,2,3,4-oxatriazolyl, 1,2,3,5-oxatriazolyl, 1,3,5-triazinyl, 1,2,4-triazinyl, 1,2,3-triazinyl, azepinyl, oxepinyl, thiepinyl, benzofuranyl, isobenzofuranyl, thionaphthenyl, isothionaphthenyl, indoleninyl, 2-isobenzazolyl, 1,5-pyrindinyl, pyrano[3,4-b]pyrrolyl, isoindazolyl, indoxazinyl, benaoxazolyl, anthranilyl, quinolinyl, isoquinolinyl, cinnolinyl, quinazolinyl, naphthyridinyl, pyrido[3,4-b]pyridinyl and pyrido[3,2-b]pyridinyl, pyrido[4,3-b]pyridinyl.

Heterocyclic systems include partially and fully saturated heteroaryl derivatives. Heterocyclic systems maybe attached to another moiety via any number of carbon atoms or heteroatoms of the radical and are both saturated and unsaturated.

It is proposed the compounds defined in Formulae I-IV or a parent of one or more of these compounds induce a physiological effect on membranous and/or transmembranous structures. In particular, and not intending to limit the present invention to any one theory or mode of action, the compounds of Formulae I-IV or a parent compound thereof are considered to block or otherwise impair the function or activity of ion channels. This effect is loosely encompassed in the term “ion channel blockers”, however, the term “blockers” is not to necessitate total blockage or prevention of ion channel activity. The term “ion channel modulators” may also be used.

Reference herein after to “ion channels” includes but is not limited to the following classes of membrane-bound ion channels and all their isoforms:
Sodium Channels, including: voltage-gated Na⁺ channels; non-voltage-gated Na⁺ channels; Na⁺/H⁺ exchangers; Na⁺-glucose transporters; Na⁺/myosinositol cotransporters; Na⁺/iodide symporters; Na⁺-dependent multivitamin transporters; voltage-gated Ca²⁺ channels, which act as voltage sensors and as Ca²⁺-selective pores (this channel-type includes L-type Ca²⁺ channels which are located in skeletal muscle, brain, cardiac muscle, neuroendocrine organs and in the retina; N-type Ca²⁺ channels which are presynaptic and involved in neurotransmitter release; P-type Ca²⁺ channels which are involved in the release of neurotransmitter at neuromuscular junctions; Q-type Ca²⁺ channels; R-type Ca²⁺ channels; and T-type Ca²⁺ channels); capacitive Ca²⁺ entry channels; ligand gated Ca²⁺ entry channels (eg. Ca²⁺ transporting ATPases); intracellular Ca²⁺ channels, including: RYR1, RYR2, RYR3, nicotinic acid adenine dinucleotide phosphate (NAAP) receptors, sphingolipid receptor (EDG1) and IP3 receptors, which act as intracellular Ca²⁺ release channels; Ca²⁺ sensors; voltage-gated K⁺ channels; inward rectifier K⁺ channels; delayed rectifier K⁺ channels; Ca²⁺ sensitive K⁺ channels (high conductance, intermediate conductance and small conductance); ATP-sensitive K⁺ channels; sodium-activated K⁺ channels; cell volume sensitive K⁺ channels; type A K⁺ channels; receptor-coupled K⁺ channels.

In a preferred embodiment, the host cell ion channels that will be affected by the ion channel modulators are from the Ca²⁺/Na⁺ exchangers, Na⁺ H⁺ exchangers, ligand- and voltage-gated Ca²⁺ channels and store-operated Ca²⁺ channels.

In a preferred embodiment, the compounds encompassed in Formulae I-IV are Verapamil (Formula V), Econazole (Formula VI), Benzamil (Formula VII) and or 5-(N-ethyl-N-isopropyl)amiloride [EIPA] (Formula VIII):

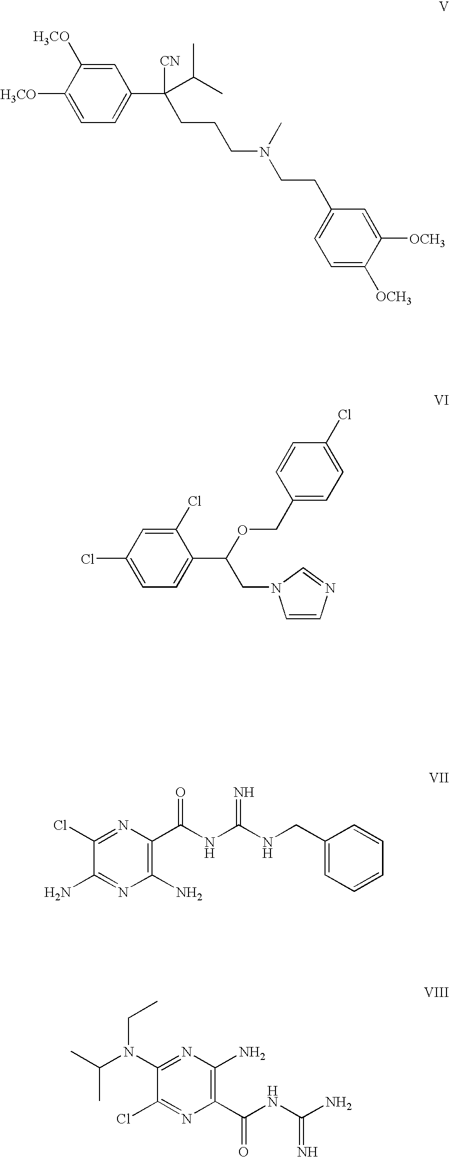

Another preferred compound is Amiloride (Formula IX) which is the parent compound of EIPA:

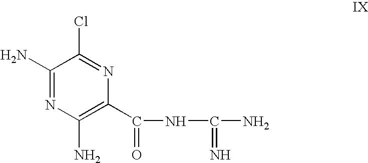

Accordingly, a preferred aspect of the present invention provides a method for ameliorating the effects of Picomaviridae infection in a vertebrate animal, said method comprising administering to said animal an effective amount of one or more compounds selected from Verapamil, Benzamil, Econazol, 5-(N-ethyl-N-isopropyl)amiloride [EIPA] and Amiloride or derivatives thereof and/or pharmaceutically acceptable salts thereof.

Reference herein to Verapamil, Benzamil, Econazol, EIPA and Amiloride include their derivatives. Examples of such derivatives are preferably compounds which fall within the scope of the compounds of general Formulae I-IV.

The vertebrate animal is as defined above and includes a human.

Accordingly, another aspect of the present invention is directed to a method for the prophylaxis or treatment of infection by a virus of the Picomaviridae family in a vertebrate animal, said method comprising administering to said animal an effective amount of a compound selected from Verapamil, Benzamil, Econazol, EIPA and Amiloride or pharmaceutically acceptable salts thereof and/or derivatives thereof wherein said derivative is selected from compounds within general Formulae I-IV, as herein defined.

Verapamil and EIPA are particularly useful against Rhinovirus.

Amiloride and Benzamil are particularly effective against Enterovirus.

The effect of these compounds is preferably but not exclusively to induce ion channel modulation and/or exhibit ion channel-modulating properties.

The terms “ion channel modulation” and “ion channel modulating properties” as contemplated herein includes an ability to alter the functional properties of an ion channel such as creating an open-channel state (complete activation), partial activation, inhibition and total blockage of the ion channel. Ion channel modulation may be induced by ion channel blockers or modulators.

The compounds may be used alone or in combination with each other and/or in combination with other ion channel blockers. Other host-cell ion-channel blocking agents contemplated by the present invention include but are not limited to: Na⁺ channel blockers: Tetrodotoxin, Saxitoxin, conotoxins, scorpion toxins, sea anemone toxins, Batrachotoxin, Ciguatoxin, Grayanotoxin, Lidocaine, Phenyloin, Amiloride, Benzamil, EIPA; Ca²⁺ channel blockers: dihydropyridines (e.g. nifedipine), phenylalkylamines (e.g. Verapamil), benzothiazepines (e.g. Diltiazem), Calciseptine, Agotoxin, SNX-325 (Segestra spider toxin), SNX-482 (Hysteroscates gigas spider toxin), nickel ions, Mibefradil, Kurtoxin, conotoxins, Econazole, EIPA; inward rectifier K⁺ channel blockers: LY97241, Gaboon viper venom, Sr²⁺, Ba²⁺, Cs²⁺; delayed rectifier K⁺ channel blockers: 4-aminopyridine, dendrotoxins, Phencyclidine, Phalloidin, 9-Aminoacridine, Margatoxin, imperator toxin, Charybdotoxin; high-conductance Ca²⁺-sensitive K⁺ channel blockers: Iberiotoxin, (+)-Tubocurarine, Charybdotoxin, Noxiustoxin, Penitrem-A, TEA; intermediate conductance Ca²⁺-sensitive K⁺ channel blockers: Cetiedil, Trifluoroperazine, Haloperidol; small conductance Ca²⁺-sensitive K⁺ channel blockers: Aparnin, Leiurotoxin 1, (+)-Tubocurarine.

Viruses contemplated in the Picornaviridae family include but are not limited to:

| Genus | Virus name (synonym) followed by (acronym) |
|---|---|
| Enterovirus | bovine enterovirus 1 (BEV-1) |
| | bovine enterovirus 2 (BEV-2) |
| | human coxsackievirus A 1 to 22 (CAV-1 to 22) |
| | human coxsackievirus A 24 (CAV-24) |
| | human coxsackievirus B 1 to 6 (CBV-1 to 6) |
| | human echovirus 1 to 7 (EV-1 to 7) |
| | human echovirus 9 (EV-9) |
| | human echovirus 11 to 27 (EV-11 to 27) |
| | human echovirus 29 to 33 (EV-29 to 33) |
| | human enterovirus 68 to 71 (HEV68 to 71) |
| | human poliovirus 1 (HPV-1) |
| | human poliovirus 2 (HPV-2) |
| | human poliovirus 3 (HPV-3) |
| | porcine enterovirus 1 to 11 (PEV-1 to 11) |
| | simian enterovirus 1 to 18 (SEV-1 to 18) |
| | Vilyuisk virus |
| Rhinovirus | bovine rhinovirus 1 (BRV-1) |
| | bovine rhinovirus 2 (BRV-2) |
| | bovine rhinovirus 3 (BRV-3) |
| | human rhinovirus 1A (HRV-1A) |
| | human rhinovirus 1 to 100 (HRV-1 to 100) |
| Hepatovirus | hepatitis A virus (HAV) |
| | simian hepatitis A virus (SHAV) |
| Cardiovirus | encephalomyocarditis virus (EMCV) |
| | (Columbia SK virus); (mengovirus) |
| | (mouse Elberfield virus) |
| | Theiler’s murine encephalomyelitis virus (TMEV) |
| | (murine poliovirus) |
| Aphthovirus | foot-and-mouth disease virus A (FMDV-A) |
| | foot-and-mouth disease virus ASIA 1 (FMDV-ASIA1) |
| | foot-and-mouth disease virus C (FMDV-C) |
| | foot-and-mouth disease virus O (FMDV-O) |
| | foot-and-mouth disease virus SAT 1 (FMDV-SAT1) |
| | foot-and-mouth disease virus SAT 2 (FMDV-SAT2) |
| | foot-and-mouth disease virus SAT 3 (FMDV-SAT3) |
| Parechovirus | Human parechovirus |
| Erbovirus | Equine rhinitis B virus |
| Kobovirus | Aichi virus |
| Teschovirus | Porcine teschovirus |

Accordingly, present invention is based on the use of substances with ion-channel blocking properties to reduce the virulence of a Picomaviridae virus, for use in the diagnosis, prophylaxis, treatment and/or symptomatic relief of Picornaviridae infections in vertebrate animals.

Preferably, the Picornaviridae virus of the present invention is from the genus Rhinovirus or Enterovirus.

Accordingly, the present invention contemplates a method for ameliorating the effects of Rhinovirus or Enterovirus infection in a vertebrate animal, said method comprising administering to said animal an effective amount of one or more compounds selected from the compounds of Formulae I-TV or a parent compound thereof.

Amiloride is an example of a parent compound of EIPA.

More particularly, the present invention contemplates a method for ameliorating the effects of Rhinovirus or Enterovirus infection in a vertebrate animal, said method comprising administering to said animal an effective amount of one or more compounds selected from the compounds of Formulae V-IX.

Even more particularly, the viral species from the genera Rhinovirus and Enterovirus are Echovirus 11 (EV11), Coxsackievirus B3 (CVB3) and Rhinovirus 2 (RV2) and Rhinovirus 14 (RV14).

As stated above, a “vertebrate animal” includes a primate, human, livestock animal (e.g. sheep, horse, cow, donkey, pig, goat), laboratory test animal (e.g. mouse, rabbit, guinea pig), companion animal (e.g. cat, dog) as well as avian, reptilian and amphibian species. The most preferred vertebrate animal is a human. The vertebrate animal of the present invention may be referred to herein as a subject.

Accordingly, the present invention contemplates a method for ameliorating the effects of Picomaviridae infection in a human subject, said method comprising administering to said human subject an effective amount of one or more compounds selected from the compounds of Formulae I-IV or a parent compound thereof.

Accordingly, the present invention contemplates a method for ameliorating the effects of Picomaviridae infection in a human subject, said method comprising administering to said human subject an effective amount of one or more compounds selected from the compounds of Formulae V-IX.

Accordingly, the present invention contemplates a method for ameliorating the effects of Rhinovirus or Enterovirus infection in a human subject, said method comprising administering to said human subject an effective amount of one or more compounds selected from the compounds of Formulae I-IV or a parent compound thereof.

Accordingly, the present invention contemplates a method for ameliorating the effects of Rhinovirus or Enterovirus infection in a human subject, said method comprising administering to said human subject an effective amount of one or more compounds selected from the compounds of Formulae I-IX or a parent compound thereof.

As used herein, “pharmaceutically acceptable salts” refer to derivatives of the disclosed compounds wherein the parent compound of any of the Formulae (I-IV) is modified by making acid or base salts of the compound of Formulae (I-IV), respectively. Examples of pharmaceutically acceptable salts include, but are, not limited to, mineral or organic acid salts of basic residues such as amines; alkali or organic salts or acidic residues such as carboxylic acids; and the like.

The compounds of the present invention may also be in the form of prodrugs.

“Prodrugs” are considered to be any covalently bonded carriers which release the active parent drug according to any of Formulae I-IV or a parent compound thereof or V-EX in vivo when such prodrug is administered to a vertebrate animal subject. Prodrugs of the compounds of Formulae I-IV or V-IX or a parent compound thereof are prepared by modifying functional groups present in the compounds in such a way that the modifications are cleaved, either in routine manipulation or in vivo, to the parent compounds. Prodrugs include compounds of Formulae I-IV wherein hydroxy, amine, or sulfhydryl groups are bonded to any group that, when administered to a subject, cleaves to form a free hydroxyl, amino or sulfhydryl group, respectively. Examples of prodrugs include, but are not limited to, acetate, formate, or benzoate derivatives of alcohol and amine functional groups in the compounds of Formulae I-IV; phosphate esters, dimethylglycine esters and carboxyalkyl esters of alcohol and phenol functional groups in the compounds of Formulae I-IV; and the like.

The pharmaceutically acceptable salts of the compounds of Formulae I-IV or a parent compound thereof include the conventional non-toxic salts or the quaternary ammonium salts of the compounds of Formulae I-IV or a parent compound thereof formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, palmoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

The pharmaceutically acceptable salts of the present invention can be synthesized from the compounds of Formulae I-IV or a parent compound thereof which contain a basic or acidic moiety by conventional chemical methods. Generally, such salts can be prepared by reacting the free acid or base forms of these compounds with a stoichiometric amount of the appropriate base or acid ill water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17^{th} ed., Mack Publishing Company, Easton, Pa., 1985, p 1418, the disclosure of which is hereby incorporated by reference.

The term “treatment” is used in its broadest sense and includes the prevention of a disease condition as well as facilitating the amelioration of the effects of the signs and symptoms of a disease.

The term “prophylaxis” is also used herein in its broadest sense to encompass a reduction in the risk of development of a disease. In certain conditions, an agent may act to treat a subject prophylactically. Furthermore, the prophylactic administration of an agent may result in the agent becoming involved in the treatment of the disease condition. Use of the terms “treatment” or “prophylaxis” is not to be taken as limiting the intended result which is to reduce the adverse effects of a disease or to potentiate the immune system's response or components therein to ameliorate the signs and/or symptoms or risk of development of the signs and/or symptoms cause or facilitated by a disease.

The present invention further extends to pharmaceutical compositions useful in the treatment of a disease condition comprising the chemical compound or compounds of the present invention. In this regard, the chemical agents of the invention can be used as actives for the treatment or prophylaxis of a disease condition such as rhinovirus. The chemical agents can be administered to a subject either by themselves, or in pharmaceutical compositions where they are mixed with a suitable pharmaceutically acceptable carrier.

An effective amount is administered. An effective amount includes a therapeutically effective amount which is an amount effective to inhibit, reduce or otherwise retard viral replication, processing and/or attachment or release. In one embodiment, the therapeutically effective amount is an amount which inhibits, blocks or at least partially impairs an ion channel. The effective amount, therefore, may be an ion channel blocking effective amount. The ability for a compound to block an ion channel may be readily observed by the downstream effect on viral replication and/or processing.

Accordingly, the invention also provides a composition for treatment and/or prophylaxis of viral infection comprising one or more chemical compounds as defined herein by Formulae I-IV or a parent compound thereof, together with one or more pharmaceutically acceptable carriers and/or diluents.

Depending on the specific conditions being treated, chemical agents may be formulated and administered systemically or locally. Techniques for formulation and administration may be found in Remington's Pharmaceutical Sciences, (supra). Suitable routes may, for example, include oral, rectal, transmucosal, or intestinal administration; nasal spray, aerosol delivery, parenteral delivery, including intramuscular, subcutaneous, intramedullary injections, as well as intrathecal, direct intraventricular, intravenous, intraperitoneal, intranasal, or intraocular injections. For injection, the chemical agents of the invention may be formulated in aqueous solutions, preferably in physiologically compatible buffers such as Hanks' solution, Ringer's solution, or physiological saline buffer. For transmucosal administration, penetrants appropriate to the barrier to be permeated are used in the formulation. Such penetrants are generally known in the art. Intra-muscular and subcutaneous injection is appropriate, for example, for administration of immunomodulatory compositions and vaccines.

The chemical agents can be formulated readily using pharmaceutically acceptable carriers well known in the art into dosages suitable for oral administration. Such carriers enable the compounds of the invention to be formulated in dosage forms such as tablets, pills, capsules, liquids, gels, syrups, slurries, suspensions and the like, for oral ingestion by a patient to be treated. These carriers may be selected from sugars, starches, cellulose and its derivatives, malt, gelatine, talc, calcium sulphate, vegetable oils, synthetic oils, polyols, alginic acid, phosphate buffered solutions, emulsifiers, isotonic saline, and pyrogen-free water.

Pharmaceutical compositions suitable for use in the present invention include compositions wherein the active ingredients are contained in an effective amount to achieve their intended purpose. The dose of agent administered to a patient should be sufficient to effect a beneficial response in the patient over time such as a reduction in the symptoms associated with the presence of an inflammatory condition in a subject. The quantity of the agent(s) to be administered may depend on the subject to be treated inclusive of the age, sex, weight and general health condition thereof. In this regard, precise amounts of the agent(s) for administration will depend on the judgement of the practitioner. In determining the effective amount of the chemical agent to be administered in the treatment or prophylaxis of a disease condition, the physician may evaluate progression of the disorder. In any event, those of skill in the art may readily determine suitable dosages of the chemical agents of the invention.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinyl-pyrrolidone (PVP). If desired, disintegrating agents may be added, such as the cross-linked polyvinyl pyrrolidone, agar, or alginic acid or a salt thereof such as sodium alginate. Such compositions may be prepared by any of the methods of pharmacy but all methods include the step of bringing into association one or more chemical agents as described above with the carrier which constitutes one or more necessary ingredients. In general, the pharmaceutical compositions of the present invention may be manufactured in a manner that is itself known, e.g. by means of conventional mixing, dissolving, granulating, dragee-making, levigating, emulsifying, encapsulating, entrapping or lyophilising processes.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical compositions which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added.

Dosage forms of the chemical agents of the invention may also include injecting or implanting controlled releasing devices designed specifically for this purpose or other forms of implants modified to act additionally in this fashion. Controlled release of an agent of the invention may be effected by coating the same, for example, with hydrophobic polymers including acrylic resins, waxes, higher aliphatic alcohols, polylactic and polyglycolic acids and certain cellulose derivatives such as hydroxypropylmethyl cellulose. In addition, controlled release may be effected by using other polymer matrices, liposomes and/or microspheres.

For any chemical agent used in the method of the invention, the therapeutically effective dose can be estimated initially from cell culture assays such as to reduce or ameliorate the symptoms of infection in vitro or to potentiate immune cells in vitro. For example, a dose can be formulated in animal models to achieve a circulating concentration range that includes the IC50 as determined in cell culture (e.g. the concentration of a test agent, which achieves a half-maximal inhibition of infection). Such information can be used to more accurately determine useful doses in humans.

Toxicity and therapeutic efficacy of such chemical agents can be determined by standard pharmaceutical procedures in cell cultures or experimental animals, e.g. for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit large therapeutic indices are preferred. The data obtained from these cell culture assays and animal studies are used in formulating a range of dosages for use in humans. The dosage of such compounds lies preferably within a range of circulating concentrations that include the ED50 with little or no toxicity. The dosage may vary within this range depending upon the dosage form employed and the route of administration utilized. The exact formulation, route of administration and dosage can be chosen by the individual physician in view of the patient's condition (see for example Fingl et al., In: The Pharmacological Basis of Therapeutics, Ch. 1 p1, 1975).

Dosage amount and interval may be adjusted individually to provide plasma levels of the active agent which are sufficient to maintain symptom-ameliorating effects. Usual patient dosages for systemic administration range from 1-2000 mg/day, commonly from 1-250 mg/day, and typically from 10-150 mg/day. Stated in terms of patient body weight, usual dosages range from 0.02-25 mg/kg/day, commonly from 0.02-3 mg/kg/day, typically from 0.2-1.5 mg/kg/day. Stated in terms of patient body surface areas, usual dosages range from 0.5-1200 mg/m²/day, commonly from 0.5-150 mg/m²/day, typically from 5-100 mg/m²/day.

Alternately, one may administer the compound in a local rather than systemic manner, for example, via injection of the compound directly into a tissue, often in a depot or sustained release formulation. Furthermore, one may administer the drug in a targeted drug delivery system, for example, in a liposome coated with tissue-specific antibody. The liposomes will be targeted to and taken up selectively by the tissue. In cases of local administration or selective uptake, the effective local concentration of the agent may not be related to plasma concentration.

The chemical agents of the invention can also be delivered topically. For topical administration, a composition containing between 0.001-5% or more chemical agent is generally suitable. Regions for topical administration include the skin surface and also mucous membrane tissues of the vagina, rectum, nose, mouth, and throat. Compositions for topical administration via the skin and mucous membranes should not give rise to signs of irritation, such as swelling or redness.

The topical composition may include a pharmaceutically acceptable carrier adapted for topical administration. Thus, the composition may take the form of a suspension, solution, ointment, lotion, sexual lubricant, cream, foam, aerosol, spray, suppository, implant, inhalant, tablet, capsule, dry powder, syrup, balm or lozenge, for example. Methods for preparing such compositions are well known in the pharmaceutical industry.

In one embodiment, the topical composition is administered topically to a subject, e.g. by the direct laying on or spreading of the composition on the epidermal or epithelial tissue of the subject, or transdermally via a “patch”. Such compositions include, for example, lotions, creams, solutions, gels and solids. Suitable carriers for topical administration preferably remain in place on the skin as a continuous film, and resist being removed by perspiration or immersion in water. Generally, the carrier is organic in nature and capable of having dispersed or dissolved therein a chemical agent of the invention. The carrier may include pharmaceutically-acceptable emollients, emulsifiers, thickening agents, solvents and the like.

The present invention is described with reference to the following non-limiting Example.

### EXAMPLE

Representative viruses of the Rhinovirus and Enterovirus genera were chosen for study: Rhinovirus 2 (RV2), Rhinovirus 14 (RV14), Coxsackievirus BE (CVB3) and Echovirus 11 (EV11). Each virus is propogated in two different cell lines as follows: RV2 and RV14 in HEL and HeLa cells, CVB3 in BS-C-1 and HeLa (human cervical adenocarcinoma) cells, EV11 in BS-C-1 (African green monkey kidney, primary) and HEL (human embryonic lung, primary) cells. Propogation of virus strains in two different cell lines is performed as different cell types may utilize different ion transport pathways.

Cells are infected with low levels of virus (0.01 plaque forming unit/cell) to obtain multiple infection cycles in the course of an experiment. Multiple infection cycles allow the detection of antiviral activity of a compound regardless of what step of the infection cycle it affects. Cells inoculated with the viruses are incubated in culture media containing different concentrations of the test compounds and dose-response curves are obtained for all compound/virus/cell type combinations. Virus yields are measured by plaque assay at the end of the experiment, and the reduction of virus yield in compound-treated samples compared with untreated cells is calculated. Cytotoxicity of the compounds is measured in parallel experiments of uninfected cells using the metabolic dye Alamar Blue as an indicator.

Antiviral assays are determined as follows. Monolayers of HeLa T cells (human cervical adenocarcinoma) in 12-well plates are infected with 0.01 plaque forming units per cell of Rhinovirus 2 in minimum essential medium with Earle's salts (MEM) supplemented with 1% v/v fetal bovine serum (FBS) or mock-infected with medium for 1 hour. The inoculum is then replaced with fresh medium containing the ion transport blockers (Verapamil, 5-(N-ethyl-N-isopropyl)amiloride [EIPA], Econazole, Benzamil or Amiloride) in concentrations ranging from 550 μM to 0 μM (no drug control) in 2-fold dilutions. Cells are further incubated for 70 hours at 34° C., until extensive cell death is observed in the no drug control. Cells plus culture supernatants are freeze-thawed and virus titre in each sample is determined by plaque assay.

As a control, the cytotoxicity of ion transport blockers is tested on cells.

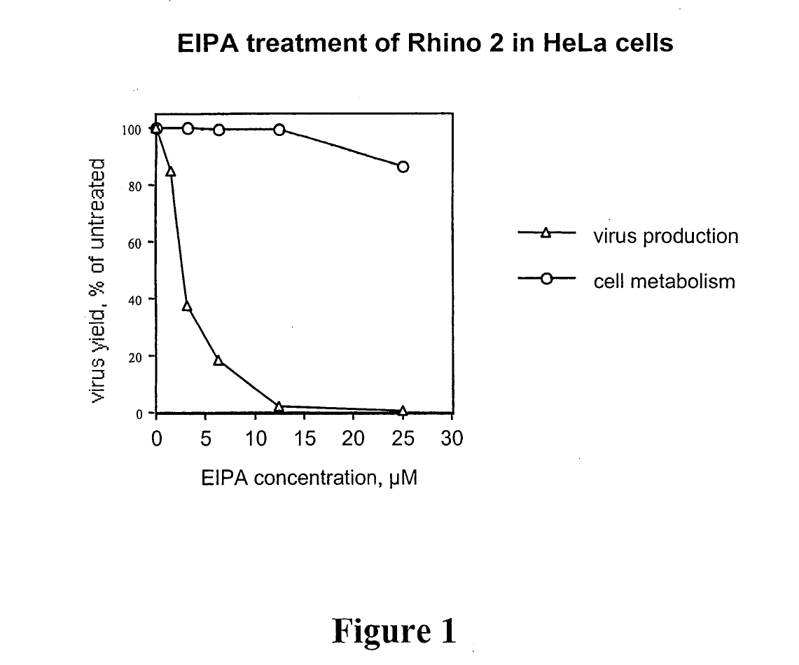

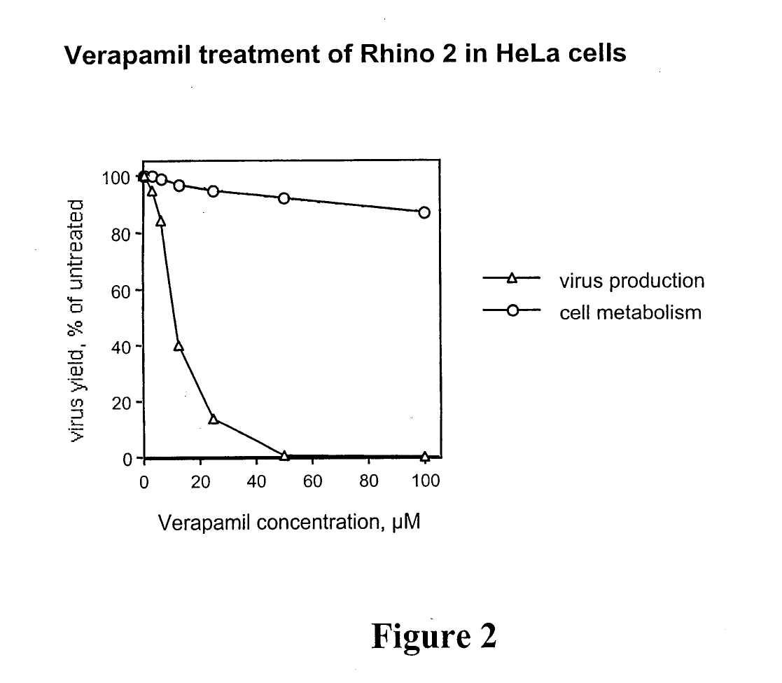

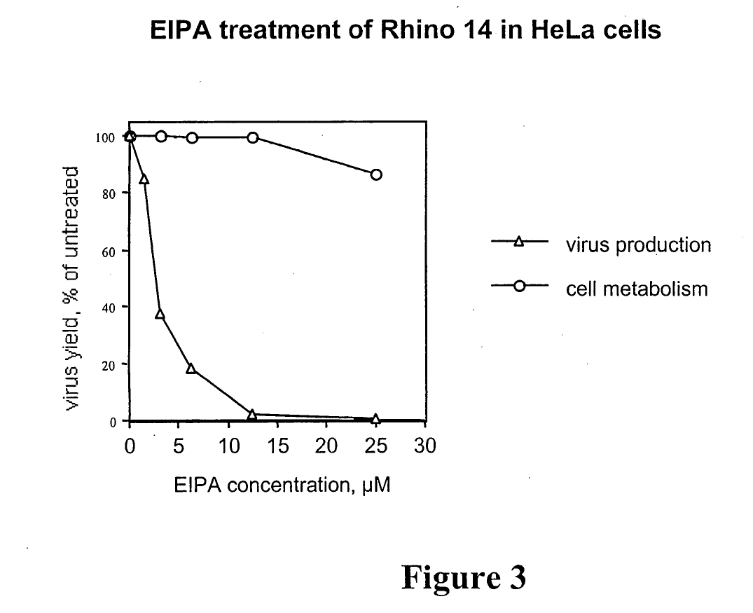

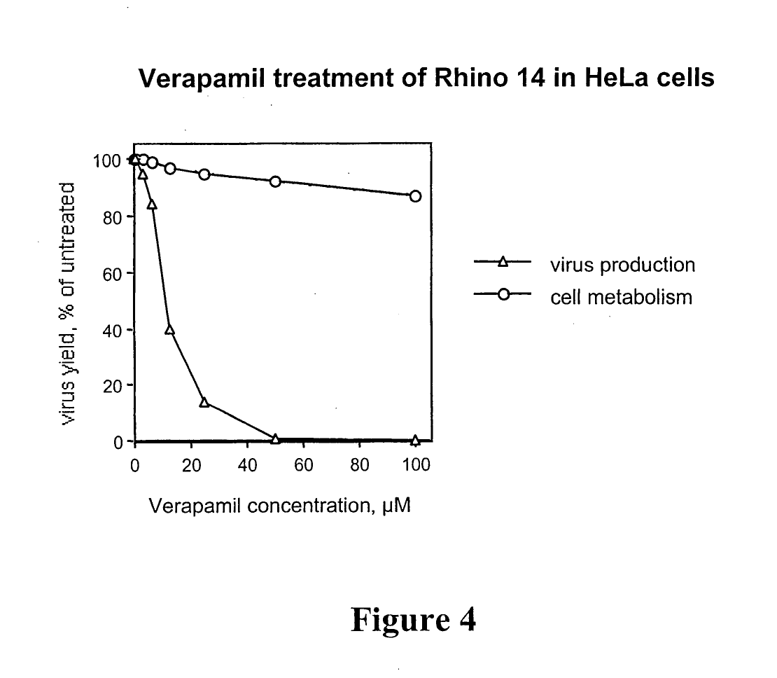

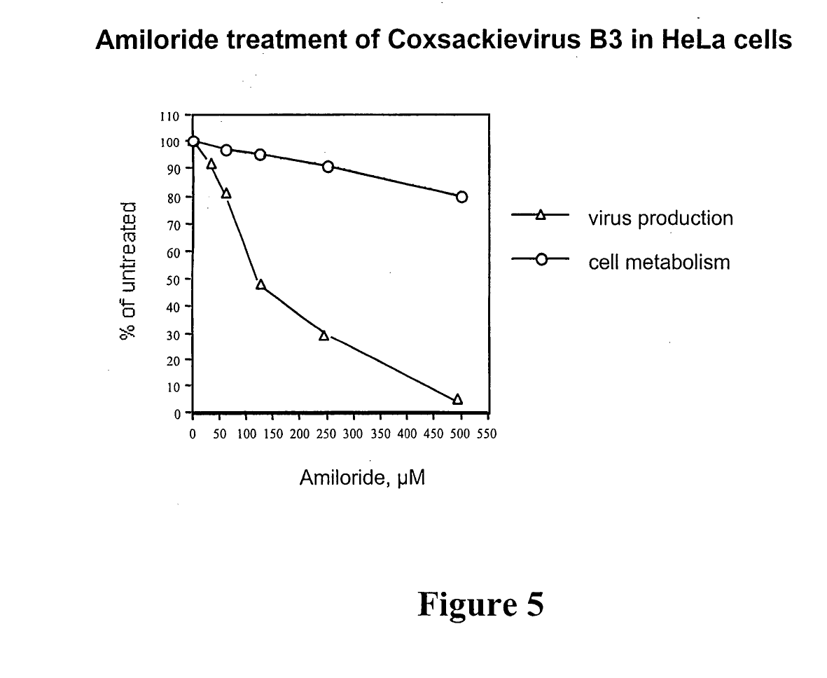

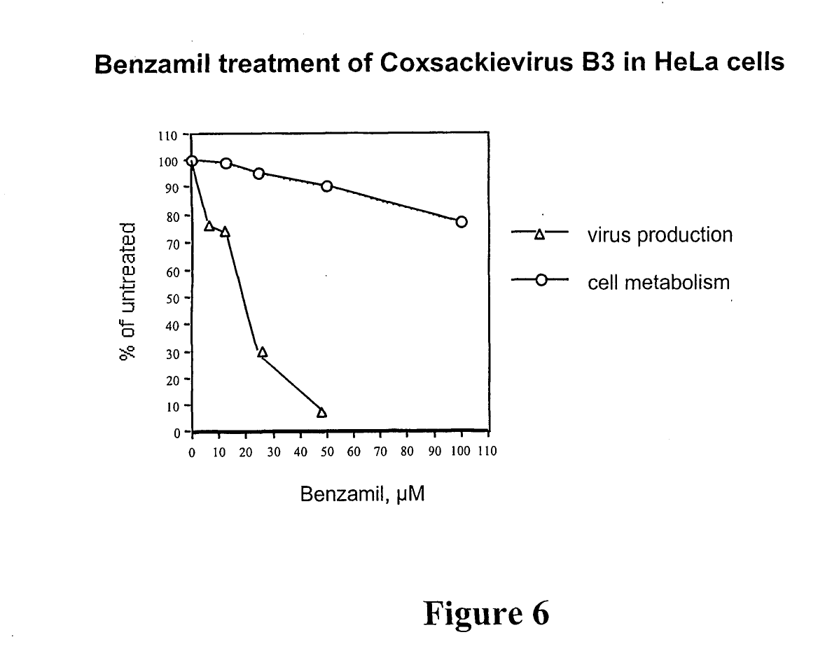

Toxicity of the compounds on HeLa T cells is evaluated in an experiment which runs parallel to the effect on viral production, using uninfected cells. Cells are incubated in MEM (1% v/v FBS) containing the ion transport blockers: Verapamil, 5-(N-ethyl-N-isopropyl)amiloride [EIPA], Econazole, Benzamil or Amiloride in concentrations ranging from 550 μM to 0 μM for 70 hours at 34° C. inside 12-well plates. The cells are then rinsed with 10 mM Tris-HCl, 150 mM NaCl, pH 7.5, and incubated for 1 hour at 34° C. in 500 μl/well of 10% solution of colourimetric indicator of metabolic activity Alamar Blue (Serotec) in MEM (1% v/v FBS). Following this step, the absorbance (A570-A600) of culture supernatants was read on a spectrophotometer (Pharmacia Biotech). Under these conditions the absorbance values are proportional to the metabolic activity of cells in each sample. The effects of EIPA and Verapamil on HeLa cells are shown in FIGS. 1 and 2 and FIGS. 3 and 4, respectively. Compared to the inhibition by these compounds of virus production, the compounds were far less toxic to HeLa cells. The effects of Amiloride and Benzamil on Coxsackievirus B3 (an Enterovirus) in HeLa cells are shown in FIGS. 5 and 6.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of anly two or more of said steps or features.

### BIBLIOGRAPHY

Monto et al., Clin. Ther. 10: 1615-27, 2001;
Turner, Antiviral Res. 49(1): 1-14, 2001;
Irurzum et al., J Virol. 69(8): 5142-6, 1995;
van Kuppeveld et al., EMBO J 16(12): 3519-32,1997;

1. A method for controlling spread of Picomaviridae infection in a vertebrate animal said method comprising administering to said animal an effective amount of one or more compounds selected from the compounds of Formulae I-IV or a parent of these compounds: 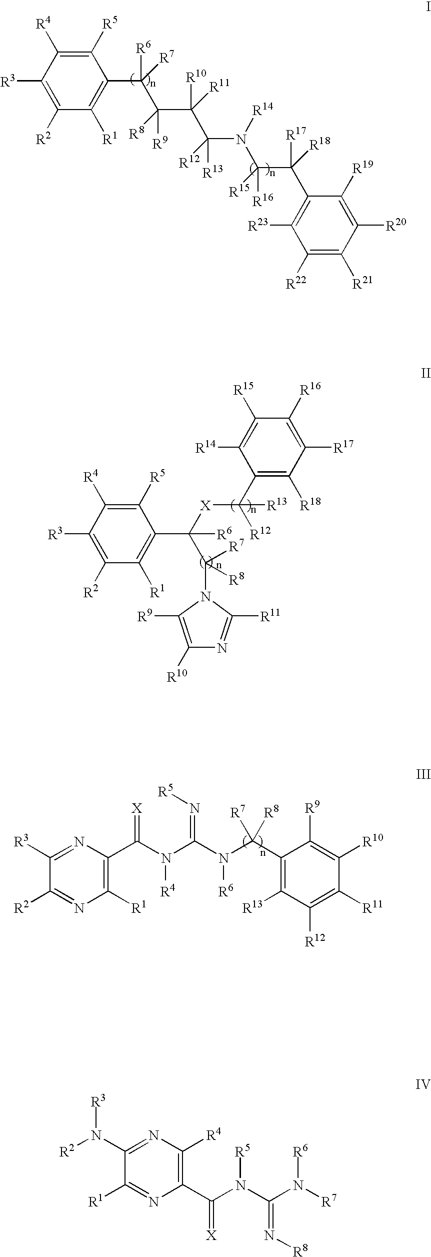
where n is 0-10 atoms and both n and X may be the same or different and each is selected from carbon, oxygen, nitrogen, sulfur, phosphorus, silicon, boron, arsenic and selenium;
R₁ to R₂₃ may be the same or different and each is selected from hydrogen, F, Cl, Br, I, CN, NC, NO₂, CF₃, COR₁, CO₂R₁, OR₁, SR₁, NR₁R₂, N(═O)₂, NR₁OR₂, ONR₁R₂, SOR₁, SO₂R₁, SO₃R₁, SONR₁R₂, SO₂NR₁R₂, SO₃NR₁R₂, P(R₁)₃, P(═O)(R₁)₃, Si(R₁)₃, B(R₁)₂, (C═X)R₁ or X(C═X)R₁ where X is selected from sulfur, oxygen and nitrogen; C₁-C₂₀ alkyl (branched and/or straight chained), C₁-C₂₀ arylalkyl, C₃-C₈ cycloalkyl, C₁-C₁₀ aldoxy, C₁-C₁₀ alkyl carbonyl, C₆-C₁₄ aryl, C₁-C₁₄ heteroaryl, C₁-C₁₄ heterocycle, C₂-C₁₀ alkenyl, C₁-C₁₀ heteroarylalkyl, C₁-C₁₀ alkoxyalkyl, C₁-C₁₀ haloalkyl, dihaloalkyl, trihaloalkyl, haloalkoxy, C₁-C₁₀ [CN, NC, OR₁, SR₁, NR₁R₂, N(═O)₂, NR₁OR₂, ONR₁R₂, SOR₁, SO₂R₁, SO₃R₁, SONR₁R₂, SO₂NR₁R₂, SO₃NR₁R₂, P(R₁)₃, P(═O)(R₁)₃, Si(R₁)₃, B(R₁)₂]alkyl; aryl is C₆-C₁₄ with any mode of substitution containing F, Cl, Br, I, NO₂, CF₃, CN, NC, COR₁, CO₂R₁, OR₁, SR₁, NR₁R₂, N(═O)₂, NR₁OR₂, ONR₁R₂, SOR₁, SO₂R₁, SO₃R₁, SONR₁R₂, SO₂NR₁R₂, SO₃NR₁R₂, P(R₁)₃, P(═O)(R₁)₃, Si(R₁)₃, B(R₁)₂]alkyl. Heteroaryl is oxazolyl, thiazaoyl, thienyl, furyl, 1-isobenzofuranyl, 3H-pyrrolyl, 2H-pyrrolyl, N-pyrrolyl, imidazolyl, pyrazolyl, isothiazolyl, isooxazolyl, pyridyl, pyrazinyl, pyrimidinyl, pyradazinyl, indolizinyl, isoindolyl, indoyl, indolyl, purinyl, phthalazinyl, 1,2,3-triazolyl, 1,2,4-triazolyl, 1,2,3-oxadiazoyl, 1,2,4-oxadiazolyl, 1,2,5-oxadiazolyl, 1,3,4-oxadiazolyl, 1,2,3,4-oxatriazolyl, 1,2,3,5-oxatriazolyl, 1,3,5-triazinyl, 1,2,4-triazinyl, 1,2,3-triazinyl, azepinyl, oxepinyl, thiepinyl, benzofuranyl, isobenzofuranyl, thionaphthenyl, isothionaphthenyl, indoleninyl, 2-isobenzazolyl, 1,5-pyrindinyl, pyrano[3,4-b]pyrrolyl, isoindazolyl, indoxazinyl, benzoxazolyl, anthranilyl, quinolinyl, isoquinolinyl, cinnolinyl, quinazolinyl, naphthyridinyl, pyrido[3,4-b]pyridinyl, pyrido[3,2-b]pyridinyl, pyrido[4,3-b]pyridinyl.

2. The method of claim 1 wherein the compounds are administered for a time and under conditions sufficient to reduce the amount of virus replication and/or release from cells exposed to said compounds.

3. The method of claim 1 or 2 wherein the compounds are administered with a pharmaceutically acceptable counterion.

4. The method of claim 3 wherein the counterion is selected from hydrochloric acid, sulphuric acid, acetic acid, lactic acid, tartaric acid, malic acid and succinic acid.

5. The method of claim 1 wherein the compounds block an ion transport pathway.

6. The method of claim 1 wherein the compound is Verapamil or a functional derivative thereof.

7. The method of claim 1 wherein the compound is Econazole or a functional derivative thereof.

8. The method of claim 1 wherein the compound is Benzamil or a functional derivative thereof.

9. The method of claim 1 wherein the compound is 5-(N-ethyl-N-iospropyl) amiloride (EIPA) or a functional derivative thereof.

10. The method of claim 1 wherein the compound is Amiloride or a functional derivative thereof.

11. The method of claim 1 or 6 or 7 or 8 or 9 or 10 wherein the vertebrate animal is a mammal.

12. The method of claim 11 wherein the mammal is a human.

13. The method of claim 1 wherein the Picornaviridae virus is Rhinovirus.

14. The method of claim 1 wherein the Picornaviridae virus is Enterovirus.

15. The method of claim 13 wherein the Rhinovirus is selected from bovine rhinovirus 1, 2 and 3, human rhinovirus 1A and human rhinovirus 1 to 100.

16. The method of claim 14 wherein the Enterovirus is selected from bovine enterovirus 1 and 2, human Coxsackievirus A1 to 22, human Coxsackievirus A24, human Coxsackievirus B1 to 6, human echovirus 1 to 7, 9, 11 to 27 and 29 to 33, human enterovirus 68 to 71, human poliovirus 1, 2 and 3, porcine enterovirus, simian enterovirus 1 to 18 and Vilyuisk virus.

17. Use of a compound which blocks an ion transport pathway which pathway results in an influx of ions into a cell following infection with a Picornaviridae virus in the manufacture of a medicament for the treatment of viral infection by a Picomaviridae virus in a vertebrate animal.

18. Use of claim 17 wherein the vertebrate animal is a mammal.

19. Use of claim 18 wherein the mammal is a human.

20. Use of claim 17 or 18 or 19 wherein the compound is selected from a compound of Formula I-IV or a parent of these compounds: 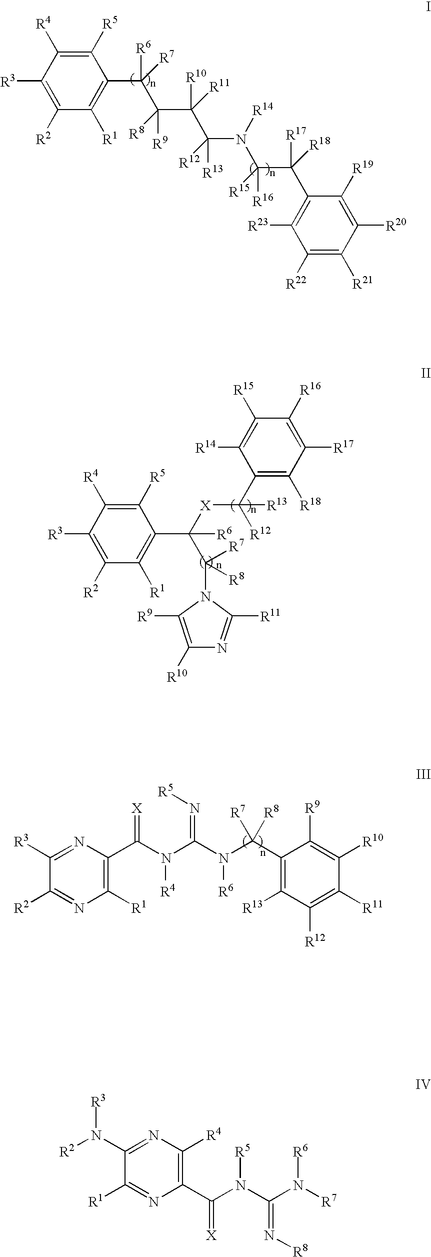
where n is 0-10 atoms and both n and X may be the same or different and each is selected from carbon, oxygen, nitrogen, sulfur, phosphorus, silicon, boron, arsenic and selenium;
R₁ to R₂₃ may be the same or different and each is selected from hydrogen, F, Cl, Br, I, CN, NC, NO₂, CF₃, COR₁, CO₂R₁, OR₁, SR₁, NR₁R₂, N(═O)₂, NR₁OR₂, ONR₁R₂, SOR₁, SO₂R₁, SO₃R₁, SONR₁R₂, SO₂NR₁R₂, SO₃NR₁R₂, P(R₁)₃, P(═O)(R₁)₃, Si(R₁)₃, B(R₁)₂, (C═X)R₁ or X(C═X)R₁ where X is selected from sulfur, oxygen and nitrogen; C₁-C₂₀ alkyl (branched and/or straight chained), C₁-C₂₀ arylalkyl, C₃-C₈ cycloalkyl, C₁-C₁₀ aldoxy, C₁-C₁₀ alkyl carbonyl, C₆-C₁₄ aryl, C₁-C₁₄ heteroaryl, C₁-C₁₄ heterocycle, C₂-C₁₀ alkenyl, C₁-C₁₀ heteroarylalkyl, C₁-C₁₀ alkoxyalkyl, C₁-C₁₀ haloalkyl, dihaloalkyl, trihaloalkyl, haloalkoxy, C₁-C₁₀ [CN, NC, OR₁, SR₁, NR₁R₂, N(═O)₂, NR₁OR₂, ONR₁R₂, SOR₁, SO₂R₁, SO₃R₁, SONR₁R₂, SO₂NR₁R₂, SO₃NR₁R₂, P(R₁)₃, P(═O)(R₁)₃, Si(R₁)₃, B(R₁)₂]alkyl; aryl is C₆-C₁₄ with any mode of substitution containing F, Cl, Br, 1, NO₂, CF₃, CN, NC, COR₁, CO₂R₁, OR₁, SR₁, NR₁R₂, N(═O)₂, NR₁OR₂, ONR₁R₂, SOR₁, SO₂R₁, SO₃R₁, SONR₁R₂, SO₂NR₁R₂, SO₃NR₁R₂, P(R₁)₃, P(═O)(R₁)₃, Si(R₁)₃, B(R₁)₂]alkyl. Heteroaryl is oxazolyl, thiazaoyl, thienyl, furyl, 1-isobenzofuranyl, 3H-pyrrolyl, 2H-pyrrolyl, N-pyrrolyl, imidazolyl, pyrazolyl, isothiazolyl, isooxazolyl, pyridyl, pyrazinyl, pyrimidinyl, pyradazinyl, indolizinyl, isoindolyl, indoyl, indolyl, purinyl, phthalazinyl, 1,2,3-triazolyl, 1,2,4-triazolyl, 1,2,3-oxadiazoyl, 1,2,4-oxadiazolyl, 1,2,5-oxadiazolyl, 1,3,4-oxadiazolyl, 1,2,3,4-oxatriazolyl, 1,2,3,5-oxatriazolyl, 1,3,5-triazinyl, 1,2,4-triazinyl, 1,2,3-triazinyl, azepinyl, oxepinyl, thiepinyl, benzofuranyl, isobenzofuranyl, thionaphthenyl, isothionaphthenyl, indoleninyl, 2-isobenzazolyl, 1,5-pyrindinyl, pyrano[3,4-b]pyrrolyl, isoindazolyl, indoxazinyl, benzoxazolyl, anthranilyl, quinolinyl, isoquinolinyl, cinnolinyl, quinazolinyl, naphthyridinyl, pyrido[3,4-b]pyridinyl, pyrido[3,2-b]pyridinyl, pyrido[4,3-b]pyridinyl.

21. Use of claim 20 wherein the compound is selected from Verapamil, Benzamil, Econazol, EIPA and Amiloride or a derivative thereof or a pharmaceutically acceptable salt thereof.

22. Use of claim 21 wherein the Picomaviridae virus is Rhinovirus or Enterovirus.

23. A method for controlling spread of Rhinovirus or Enterovirus infection in a vertebrate animal, said method comprising administering to said animal an effective amount of one or more compounds selected from the compounds of Formulae I-IV or a parent compound thereof.

24. The method of claim 23 wherein the compound is selected from Verapamil, Benzamil, Econazol, EIPA and Amiloride.

25. The method of claim 23 or 24 wherein the vertebrate animal is a mammal.

26. The method of claim 25 wherein the mammal is a human.

27. A pharmaceutical preparation when used to control spread of Picomaviridae infection according to the method of claim 1 comprising one or more compounds selected from Verapamil, Benzamil, Econazol, EIPA and Amiloride or a pharmaceutically acceptable salt thereof and a pharmaceutically acceptable carrier and/or diluent.
